(19) United States
(12) Patent Application Publication
(10) Pub. No.: US 2021/0204819 A1
(43) Pub. Date: Jul. 8, 2021

(54) PATCH TYPE THERMOMETER-BASED BODY TEMPERATURE MANAGEMENT SYSTEM AND METHOD THEREOF

(71) Applicants: AMOTECH CO., LTD., Incheon (KR); MOBILE DOCTOR CO., LTD, Seoul (KR)

(72) Inventors: Beom Jin KIM, Bucheon-si (KR); Se Yun CHANG, Seoul (KR); Nam Soo OH, Seongnam-si (KR)

(21) Appl. No.: 17/269,066

(22) PCT Filed: Aug. 16, 2019
(86) PCT No.: PCT/KR2019/010407

§ 371 Date: Feb. 17, 2021

(30) Foreign Application Priority Data
Aug. 21, 2018 (KR) 10-2018-0097397

(51) Int. Cl. A61B 5/01; A61B 5/00; A61B 5/117

(52) U.S. Cl. CPC A61B 5/01; A61B 5/6833; A61B 5/0008; A61B 2560/0223; A61B 5/117; A61B 2562/0271; A61B 2562/166; A61B 5/742

(57) ABSTRACT

A patch type thermometer-based body temperature management system includes: a patch type thermometer which is attached to the skin of a user to measure body temperature, and transmits a user ID, related application (App) information, and body temperature data on the basis of NFC; a terminal configured to form a magnetic field by tagging the patch type thermometer, to request a body temperature measurement, and to receive the user ID, the related App information, and the body temperature data from the patch type thermometer to calculate a body temperature value; and a body temperature management server configured to receive the body temperature value from the terminal, accumulate and store the received body temperature value to calculate at least one of trend of changes in body temperature over time and a state of health according to the received body temperature value and transmit same to the terminal.

### TECHNICAL FIELD

The present invention relates to a patch-type thermometer-based body temperature management system and method.

### BACKGROUND ART

Generally, as methods of measuring a body temperature, there are methods of using a contact type clinical thermometer and methods of using a non-contact type clinical thermometer, and examples of general clinical thermometers include mercury thermometers, alcohol thermometers, infrared sensors, and the like.

Such a clinical thermometer may measure body temperature only by waiting for a period of time while being worn or in contact with a body.

Accordingly, in the case of patients such as infants, toddlers, and people with reduced mobility, there is inconvenience in measuring a body temperature because people around the patient need to help the patient keep the clinical thermometer on a body of the patient.

In addition, there is inconvenience in checking the body temperature in real time or periodically because the conventional clinical thermometer uses a method of checking the body temperature by separating the clinical thermometer from a body of a user after being brought into contact with the body of the user for a predetermined period of time.

In addition, in order to manage measured body temperature information, there is inconvenience in that the user should input the measured body temperature information and transmit the measured body temperature information to a management server whenever the body temperature is measured.

### DISCLOSURE

### Technical Problem

The present invention is directed to providing a patch-type thermometer-based body temperature management system and method, in which a patch-type thermometer is attached to a body of a user and a body temperature is easily measured and, at the same time, a calculated body temperature value is automatically transmitted to a body temperature management server and managed.

The present invention is also directed to providing a patch-type thermometer-based body temperature management system and method, in which a health condition of a user is provided based on measured body temperature information and an action is provided according to the health condition.

The present invention is also directed to providing a patch-type thermometer-based body temperature management system and method, in which pieces of measured body temperature information are accumulated and stored so that a trend of a change in body temperature for each preset period is provided.

### Technical Solution

One aspect of the present invention provides a patch-type thermometer-based body temperature management system including a patch-type thermometer, a terminal, and a body temperature management server. The patch-type thermometer is attached to skin of a user to measure body temperature and transmits a user identification (ID), related app information, and body temperature data on the basis of near-field communication (NFC). The terminal forms a magnetic field by being tagged to the patch-type thermometer, requests measurement of the body temperature, receives the user ID, the related app information, and the body temperature data from the patch-type thermometer, and calculates body temperature values. The body temperature management server receives the body temperature values from the terminal, accumulates and stores the received body temperature values, calculates at least one of a trend of a change in body temperature over time and a health condition corresponding to the received body temperature values, and transmits the calculated trend of the change or health condition to the terminal.

The terminal may include a body temperature calculating unit and a body temperature information processing unit. The body temperature calculating unit may calculate the body temperature values using the body temperature data received from the patch-type thermometer. The body temperature information processing unit may control the body temperature values to be transmitted to the body temperature management server, the trend of the change in body temperature for the accumulated body temperature values and the health condition to be requested to the body temperature management server, and corresponding information to be received and displayed.

The body temperature calculating unit may receive a plurality of pieces of body temperature data from the patch-type thermometer, perform correction for the plurality of pieces of body temperature data and then calculate the body temperature values according to the corrected body temperature data, calculate an average value of the calculated body temperature values when a deviation of the calculated body temperature values is less than or equal to a predetermined value, delete a first one piece of data among the pieces of body temperature data, update the body temperature data to new data, and re-calculate the body temperature value when the deviation of the calculated body temperature values exceeds the predetermined value.

In this case, the body temperature calculating unit may correct the body temperature data on the basis of pre-calibration data for a temperature sensor of the patch-type thermometer and sequentially perform correction for internal resistance of a driving chip of the patch-type thermometer, correction for an error (a radio frequency (RF) harvesting error) that occurs when a voltage is rectified according to being inductively coupled by NFC tagging, and correction for a thermistor of the patch-type thermometer and external resistance of the driving chip to correct the body temperature data.

The terminal may further include a first communication unit, a second communication unit, a display unit, and a storage unit. The first communication unit may perform NFC with the patch-type thermometer. The second communication unit may perform communication with the body temperature management server via a wired or wireless communication network. The display unit may display the body temperature values and corresponding information received from the body temperature management server. The storage unit may store the body temperature values.

The body temperature management server may include a body temperature information management unit, a health condition determination unit, and a database. The body temperature information management unit may accumulate, store, and manage the body temperature values received from the terminal over time, and calculate a body temperature graph according to the trend of the change in body temperature. The health condition determination unit may determine a current health condition according to the received body temperature values. The database may store user information, body temperature information, and action information.

In this case, the body temperature information management unit may calculate the body temperature graph for each preset time unit. Further, the health condition determination unit may calculate an action corresponding to the determined health condition.

The patch-type thermometer may include a flexible circuit board, a temperature sensor, a heat transfer member, and a protective member. An antenna pattern may be formed and at least one driving chip is mounted on at least one surface of the flexible circuit board. The temperature sensor may be mounted on an upper surface of the flexible circuit board so as to measure the body temperature. The heat transfer member may be electrically connected to the temperature sensor through a via hole and may be mounted on a lower surface of the flexible circuit board so as to be in direct contact with the skin of the user. The protective member may surround the flexible circuit board so as to prevent the antenna pattern, the driving chip, and the temperature sensor from being exposed to the outside.

In this case, the patch-type thermometer may be driven by being inductively coupled with the magnetic field.

Another aspect of the present invention provides a patch-type thermometer-based body temperature management method including forming a magnetic field by a terminal being tagged to a patch-type thermometer attached to a body of a user, starting driving of the patch-type thermometer by being inductively coupled with the magnetic field, transmitting, by the patch-type thermometer, a user ID and related app information to the terminal, driving, by the terminal, a corresponding app according to the related app information, measuring, by the patch-type thermometer, a body temperature of the user in response to a request of the terminal and transmitting body temperature data to the terminal, and calculating, by the terminal, body temperature values according to the body temperature data.

The transmitting of the body temperature data to the terminal may include measuring the body temperature data multiple times and repeatedly transmitting the measured data. Here, the calculating of the body temperature values may include performing correction on the body temperature data transmitted multiple times, then calculating the body temperature values according to the corrected body temperature data, calculating an average value of the calculated body temperature values when a deviation of the calculated body temperature values is less than or equal to a predetermined value, deleting a first one piece of data among the pieces of body temperature data, updating the body temperature data to new data, and re-calculating the body temperature value when the deviation of the calculated body temperature value exceeds the predetermined value.

In this case, the calculating of the body temperature values may include correcting the body temperature data on the basis of pre-calibration data for a temperature sensor of the patch-type thermometer. Here, the correction of the body temperature data may include sequentially performing correction for internal resistance of a driving chip of the patch-type thermometer, correction for an error (an RF harvesting error) that occurs when a voltage is rectified according to being inductively coupled by NFC tagging, and correction for a thermistor of the patch-type thermometer and external resistance of the driving chip.

The patch-type thermometer-based body temperature management method may further include transmitting, by the terminal, the calculated body temperature values to the body temperature management server, and accumulating and storing, by the body temperature management server, the received body temperature values over time.

The patch-type thermometer-based body temperature management method may further include requesting, by the terminal, a health condition corresponding to the body temperature value from the body temperature management server, determining, by the body temperature management server, the health condition corresponding to the body temperature value, and receiving, by the terminal, the health condition from the body temperature management server and displaying the health condition.

The determining of the health condition may further include calculating an action corresponding to the determined health condition. In this case, the displaying of the health condition may include displaying the action together with the health condition.

The patch-type thermometer-based body temperature management method may further include requesting, by the terminal, a trend of a change in body temperature from the body temperature management server, calculating, by the body temperature management server, a body temperature graph for each period using the stored body temperature values, and receiving, by the terminal, the calculated body temperature graph from the body temperature management server and displaying the body temperature graph.

In this case, the calculating of the body temperature graph may include calculating the body temperature graph for each preset time unit.

### Advantageous Effects

According to the present invention, by tagging a terminal that a user or a guardian possesses to a patch-type thermometer which is in a state of being attached to skin of the user, a body temperature of the user can be easily measured and, at the same time, a calculated body temperature value can be automatically transmitted to a body temperature management server, and thus convenience of use can be improved.

Further, according to the present invention, by providing a health condition of the user on the basis of measured body temperature information and providing an action according to the health condition, even when there is no specialized knowledge, a temporary action can be rapidly performed and thus the health of the user can be stably managed.

Further, according to the present invention, by accumulating and storing pieces of measured body temperature information and providing a trend of a change in body temperature for each preset period, an accurate condition of the user can be presented to a medical staff so that the medical staff can take rapid action, and thus the treatment process can be shortened.

### DESCRIPTION OF DRAWINGS

FIG. 1 is a schematic block diagram illustrating a patch-type thermometer-based body temperature management system according to an embodiment of the present invention.

FIG. 2 is a perspective view illustrating a patch-type thermometer of FIG. 1.

FIG. 3 is a bottom view illustrating a state in which a release film of FIG. 2 is separated.

FIG. 4 is a perspective view illustrating an internal configuration of FIG. 2.

FIG. 5 is a cross-sectional view taken along line A-A of FIG. 4.

FIG. 6 is a block diagram illustrating a detailed configuration of a terminal of FIG. 1.

FIG. 7 is a diagram illustrating a data format for near-field communication (NFC) between the terminal and the patch-type thermometer of FIG. 1.

FIG. 8 is a block diagram illustrating a detailed configuration of a body temperature management server of FIG. 1.

FIG. 9 is a flowchart illustrating an example of a patch-type thermometer-based body temperature management method according to an embodiment of the present invention.

FIG. 10 is a flowchart illustrating another example of the patch-type thermometer-based body temperature management method according to the embodiment of the present invention.

FIG. 11 is a flowchart illustrating still another example of the patch-type thermometer-based body temperature management method according to the embodiment of the present invention.

FIG. 12 is a flowchart illustrating yet another example of the patch-type thermometer-based body temperature management method according to the embodiment of the present invention.

### MODES OF THE INVENTION

Hereinafter, embodiments of the present invention that can be easily performed by those skilled in the art will be described in detail with reference to the accompanying drawings. The embodiments of the present invention may be implemented in several different forms and are not limited to the embodiments described herein. Parts irrelevant to description are omitted in the drawings in order to clearly explain the present invention. The same or similar parts are denoted by the same reference numerals throughout this specification.

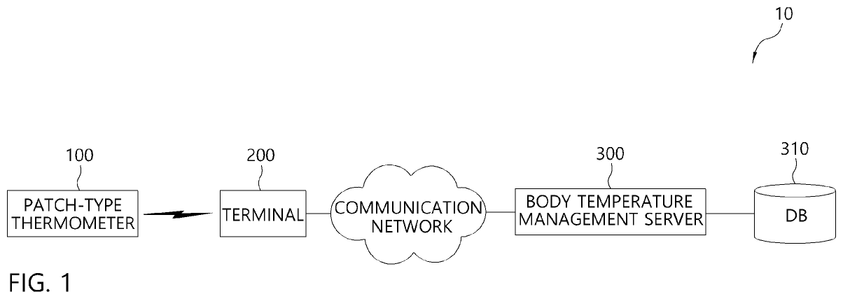

A patch-type thermometer-based body temperature management system 10 according to an embodiment of the present invention includes a patch-type thermometer 100, a terminal 200, and a body temperature management server 300, as illustrated in FIG. 1.

The patch-type thermometer 100 is attached to skin of a user to measure body temperature of the user. For example, the patch-type thermometer 100 may be driven based on near-field communication (NFC). That is, the patch-type thermometer 100 is driven by being inductively coupled with a magnetic field formed from the terminal 200 and transmits a user identification (ID), related app information, and measured body temperature data of the patch-type thermometer 100 to the terminal 200 on the basis of NFC. Here, in the patch-type thermometer 100, a temperature sensor embedded therein may be an active sensor or a passive sensor.

The terminal 200 forms a magnetic field by tagging the patch-type thermometer 100 and requests body temperature measurement from the patch-type thermometer 100. For example, the terminal 200 may communicate with the patch-type thermometer 100 on the basis of NFC. That is, the terminal 200 receives the user ID, the related app information, and the body temperature data from the patch-type thermometer 100. In this case, the terminal 200 calculates a body temperature value recognizable by the user on the basis of the body temperature data received from the patch-type thermometer 100.

Here, the terminal 200 is a terminal of a user or guardian and may be a terminal that may communicate with the patch-type thermometer 100 on the basis of NFC, drive a related app, and perform remote communication in order to communicate with the body temperature management server 300. For example, the terminal 200 may be a portable electronic device, such as a mobile phone or a tablet personal computer (PC), or a wearable device such as a smart watch, but the present invention is not limited thereto.

In this case, the terminal 200 may measure body temperature using the patch-type thermometer 100 and, at the same time, automatically transmit a calculated body temperature value to the body temperature management server 300.

The body temperature management server 300 receives and stores the calculated body temperature value from the terminal 200. In this case, the body temperature management server 300 accumulates and stores the received body temperature values over time. Here, the body temperature management server 300 calculates information requested by the terminal 200 on the basis of the stored body temperature values and transmits the calculated information to the terminal 200.

For example, the body temperature management server 300 calculates a trend of a change in the body temperature on the basis of the stored body temperature values in response to the request of the terminal 200. As another example, the body temperature management server 300 determines a health condition of the user according to the body temperature values received from the terminal 200.

Here, the body temperature management server 300 is a server for providing a body temperature management service to the users through the related app and may be a server operated by a service provider. In this case, various types of terminals may be connected to the body temperature management server 300 via a wired or wireless communication network.

As described above, by tagging the terminal 200 that the user or a manager possesses to the patch-type thermometer 100 which is in a state of being attached to the skin of the user, the body temperature of the user may be easily measured. At the same time, the measured body temperature may be transmitted to the body temperature management server 300 without special manipulation, and thus convenience of use may be improved.

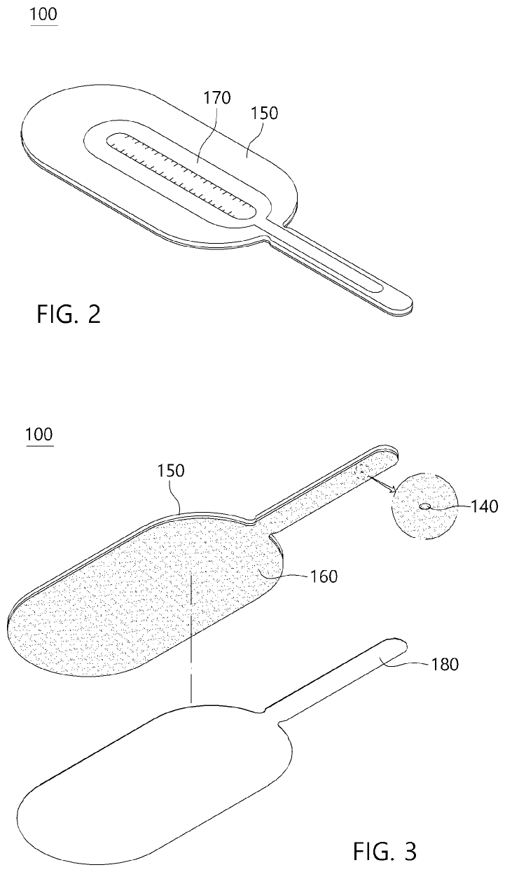

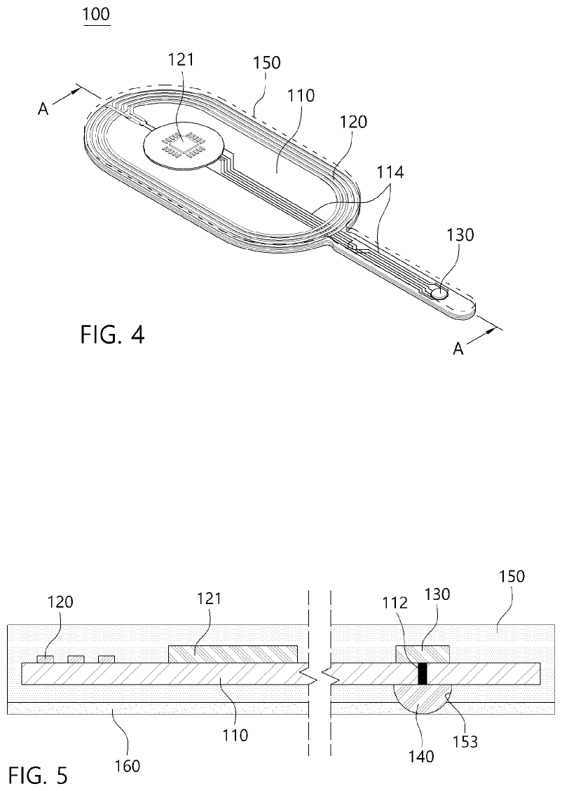

Referring to FIGS. 2 to 5, the patch-type thermometer 100 includes a flexible circuit board 110, a temperature sensor 130, a heat transfer member 140, and a protective member 150.

The flexible circuit board 110 may be a substrate on which various types of circuit elements are mounted or a circuit pattern for electrical connection is formed. For example, the circuit element may be a chipset-type element that performs a predetermined function, and the circuit pattern may be an antenna pattern or a wiring pattern for electrical connection.

The flexible circuit board 110 may be a known flexible printed circuit board (FPCB) having flexibility using polyimide (PI), polyethylene terephthalate (PET), or the like.

In this case, an antenna pattern 120 may be formed on at least one surface of the flexible circuit board 110, and at least one driving chip 121 electrically connected to the antenna pattern 120 may be mounted on the flexible circuit board 110. Further, the temperature sensor 130 may be mounted on one surface of the flexible circuit board 110 and be electrically connected to the driving chip 121 through a lead portion 114.

For example, the antenna pattern 120 may be an NFC antenna for short-range wireless communication, and the driving chip 121 may be an NFC driving chip that drives the antenna pattern 120.

Accordingly, the antenna pattern 120 is driven by the driving chip 121 mounted on the flexible circuit board 110 to serve as a radiator for transmitting information obtained through the temperature sensor 130 to the terminal 200 using an NFC communication method.

Accordingly, body temperature data measured using the temperature sensor 130 may be transmitted to the terminal 200 through the antenna pattern 120 when NFC tagging with the terminal 200 is performed.

Meanwhile, the antenna pattern 120 may serve to transmit data for transmitting the information obtained through the temperature sensor 130 and also serve to generate driving power required by the driving chip 121.

That is, the antenna pattern 120 may be inductively coupled with the magnetic field formed from the terminal 200 and may supply power generated by being inductively coupled to the driving chip 121. Specifically, the antenna pattern 120 may generate power for driving the driving chip 121 by being inductively coupled with the magnetic field formed from the terminal 200 when the NFC tagging is performed. Here, the generation of power may be referred to as radio frequency (RF) harvesting.

Accordingly, the driving chip 121 may be driven using the power received from the antenna pattern 120 when the NFC tagging is performed, and the information obtained through the temperature sensor 130 may be transmitted to the terminal 200 through the antenna pattern 120.

Accordingly, the patch-type thermometer 100 does not require separate power for driving the driving chip 121 and thus a total weight thereof may be reduced, and a battery is omitted and thus the patch-type thermometer 100 may be implemented as an ultra-thin type.

The temperature sensor 130 may generate information about the body temperature of the user by detecting the body temperature of the user. The temperature sensor 130 may be mounted on one surface of the flexible circuit board 110.

In this case, the temperature sensor 130 may be a digital temperature sensor and measure the body temperature of the user on the basis of heat which will be transmitted through the heat transfer member 140.

To this end, the temperature sensor 130 may be mounted on an upper surface of the flexible circuit board 110, the heat transfer member 140 may be mounted on a lower surface of the flexible circuit board 110 so as to be brought into direct contact with the skin of the user, and the temperature sensor 130 and the heat transfer member 140 may be electrically connected to each other through a via hole 112.

Accordingly, the temperature sensor 130 may be mounted on the upper surface of the flexible circuit board 110, which is coplanar with the driving chip 121, without being exposed to the skin of the user and may be fully covered with the protective member 150 described below, and thus airtightness may be increased.

In such a case, the driving chip 121 may be disposed inside the antenna pattern 120, the temperature sensor 130 may be disposed outside the antenna pattern 120, and the driving chip 121 of and the temperature sensor 130 may be electrically connected to each other through the lead portion 114 formed on at least one surface of the flexible circuit board 110.

In this case, the heat transfer member 140 may be mounted on the lower surface of the flexible circuit board 110 as described above and thus may be brought into direct contact with the skin of the user. Accordingly, the body temperature transmitted from the skin of the user may be transmitted to the temperature sensor 130 through the heat transfer member 140.

To this end, the heat transfer member 140 may be made of a metal material having high thermal conductivity.

In this case, the heat transfer member 140 may have a shape in which a state of being in contact with the skin of the user may always be maintained. To this end, the heat transfer member 140 may have a shape in which a central portion thereof protrudes convexly in one direction.

For example, the heat transfer member 140 may be formed to have a hemispherical shape or a dome shape.

Accordingly, when the patch-type thermometer 100 is attached to the skin of the user, a central portion of the heat transfer member 140 may be maintained in a state of always being in contact with the skin of the user even when the attachment portion is curved. Accordingly, the heat transfer member 140 may smoothly transmit heat transmitted from the skin of the user to the temperature sensor 130.

As illustrated in FIGS. 3 and 5, the heat transfer member 140 may be exposed to the outside through an exposure hole 153 formed in the protective member 150 to be described below. Accordingly, when the patch-type thermometer 100 is attached to the body of the user, the heat transfer member 140 may always be in direct contact with the skin of the user.

Meanwhile, the patch-type thermometer 100 may include the protective member 150 that surrounds the flexible circuit board 110 to prevent the antenna pattern 120, the driving chip 121, and the temperature sensor 130 from being exposed to the outside.

In such a case, in the protective member 150, the exposure hole 153 may be formed to pass through a portion corresponding to the heat transfer member 140, as described above, and thus the heat transfer member 140 may be exposed to the outside through the exposure hole 153.

Accordingly, the protective member 150 may be formed to fully surround remaining portions except for the portion corresponding to the heat transfer member 140.

That is, the protective member 150 may be disposed to fully cover the upper and lower surfaces of the flexible circuit board 110 and thus may prevent the antenna pattern 120, the driving chip 121, the temperature sensor 130, and the flexible circuit board 110, excluding the heat transfer member 140, from being exposed to the outside.

In this case, the protective member 150 may be made of a material having flexibility. Accordingly, even when the patch-type thermometer 100 is attached to a curved portion of the body, the patch-type thermometer 100 may be flexibly changed according to the curved portion of the body of the user, and thus adhesion with the body of the user may be improved.

For example, the protective member 150 may be a molding made of an insulating resin such as silicone. However, the material of the protective member 150 is not limited thereto, and the protective member 150 may be made in the form of a sheet formed of a fluoropolymer resin such as polyethylene terephthalate (PET), polypropylene (PP), or polyethylene (PE), or release paper, and may use any material having insulation and airtightness without limitation.

Meanwhile, in the patch-type thermometer 100, an adhesive layer 160 may be formed on one surface of the protective member 150.

The adhesive layer 160 may impart adhesion and thus allow the patch-type thermometer 100 to be attached to the body of the user. Here, the adhesive layer 160 may be formed on a surface in which the exposure hole 153 for exposing the heat transfer member 140 to the outside is formed. Accordingly, when the patch-type thermometer 100 is attached to the skin of the user through the adhesive layer 160, the heat transfer member 140 may be brought into direct contact with the skin of the user.

For example, the adhesive layer 160 may be a gel-type adhesive layer and may be reused repeatedly because the adhesive layer 160 is made of a material of which adhesion is restored when brought into contact with moisture.

However, the material of the adhesive layer 160 is not limited thereto, and it is noted that any material capable of providing adhesion to the skin of the user may be used as the material of the adhesive layer 160 without limitation.

Further, in the patch-type thermometer 100, an information display unit 170 may be formed on one surface of the protective member 150. The information display unit 170 may include at least one piece of information of text, numbers, and figures.

For example, the information display unit 170 may be a logo or a figure for aesthetic sense. Accordingly, the user may check various pieces of information through the information display unit 170 to identify information about a product.

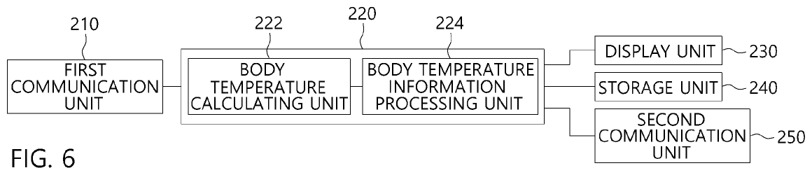

Referring to FIG. 6, the terminal 200 may include a first communication unit 210, a control unit 220, a display unit 230, a storage unit 240, and a second communication unit 250.

The first communication unit 210 communicates with the patch-type thermometer 100. For example, the first communication unit 210 communicates with the patch-type thermometer 100 in an NFC method. In this case, the first communication unit 210 may form a magnetic field using the patch-type thermometer 100 when communicating with the patch-type thermometer 100. That is, the first communication unit 210 may provide power to the patch-type thermometer 100 by being inductively coupled with the antenna pattern 120 of the patch-type thermometer 100.

Further, the first communication unit 210 may communicate with the patch-type thermometer 100 according to a data format.

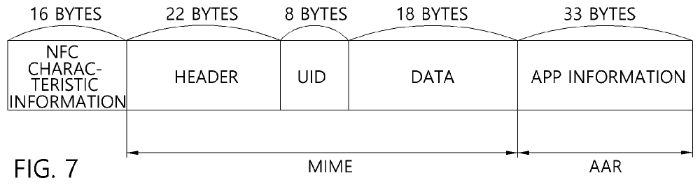

Referring to FIG. 7, the data format for NFC between the patch-type thermometer 100 and the terminal 200 includes an NFC data exchange format (NDEF).

Here, the NDEF is composed of Multipurpose Internet Mail Extensions (MIME) including measurement information of the temperature sensor 130 of the patch-type thermometer 100 and Android Application Records (AAR) for automatically executing related apps.

A first field of the NDEF format includes NFC characteristic information. Here, the NFC characteristic information includes a version in the NDEF format, the number of blocks that may be read or written at one time through a “CHECK” or “UPDATE” command, the maximum number of blocks that may use NDEF data, a write flag, attribute information such as being readable or writable, checksums, and the like.

A header of MIME data includes a length of an MIME type name, a length of a payload, the MIME type name, a firmware version as a payload, and the like. Here, the firmware version indicates whether the temperature sensor is a passive sensor or an active sensor.

UID denotes a user ID and may be a unique number of the driving chip 121 of the patch-type thermometer 100. That is, the UID is ID information about the patch-type thermometer 100.

The data includes body temperature data and correction data. Here, the body temperature data is data corresponding to first two bytes in the case of the active sensor. In this case, the body temperature data may be unsigned 16-bit data.

Further, in the case of the passive sensor, the correction data may include calibration information for correcting deviations of individual products during mass production. Here, the passive sensor may include a thermistor. That is, the passive sensor may include data necessary for correction together with the body temperature data. In this case, the body temperature data may be a measurement value for the thermistor.

The correction data corresponds to a measurement value of an analog-to-digital converter (ADC) that calculates data in the driving chip 121. Here, the correction data includes an adjusted voltage (two bytes) of the driving chip 121, a measurement value (two bytes) for a reference voltage, a measurement value (two bytes) of a rectified voltage by RF harvesting, a measurement value (two bytes) for the thermistor, a correction value (two bytes) of internal resistance of the driving chip for thermistor measurement, an error correction value (one byte) of the thermistor, a measurement value (two bytes) for external resistance, a correction value (two bytes) of internal resistance of the driving chip for external resistance measurement, an error correction value (two bytes) for external resistance, and an actual temperature value (one byte) at the time of calibration (in mass production).

The AAR may include a length of the AAR type name, a length of a payload, the AAR type name, and the payload.

The control unit 220 controls NFC communication with the patch-type thermometer 100 and communication with the body temperature management server 300 via a wired and/or wireless communication network to be performed, and controls calculating and processing the body temperature to be performed. Here, the control unit 220 may recognize that the patch-type thermometer 100 inductively coupled with the first communication unit 210 is an NFC clinical thermometer.

In this case, the control unit 220 may be provided with related apps installed in the terminal 200. That is, the control unit 220 may be provided in software driven by a processor of the terminal 200. Here, the control unit 220 may include a body temperature calculating unit 222 and a body temperature information processing unit 224.

The body temperature calculating unit 222 may calculate the body temperature data received from the patch-type thermometer 100 as a body temperature value. Here, the body temperature data may be data transmitted according to a data format, and the body temperature value may be physical temperature information that may be recognized by the user. That is, the body temperature calculating unit 222 may calculate the body temperature value by converting the body temperature data of a 16-bit value (unsigned integer) into physical scale data. For example, when the temperature sensor is an active sensor, the body temperature calculating unit 222 may calculate the body temperature value according to Equation 1 below.

Body temperature value=−C1+C2×(body temperature data)/(2¹⁶−1) [Equation 1]

Here, C1 denotes a compensation constant, and C2 denotes a linearization constant for a supply voltage. Further, when the temperature sensor is a passive sensor, the body temperature calculating unit 222 may receive a plurality of pieces of body temperature data from the patch-type thermometer 100. For example, the body temperature calculating unit 222 may determine whether five pieces of data are transmitted, and when it is determined that five pieces of data are not transmitted, the body temperature calculating unit 222 may request transmission of the body temperature data from the patch-type thermometer 100.

In this case, the body temperature calculating unit 222 may correct the plurality of pieces of body temperature data and then calculate the body temperature value according to the pieces of corrected body temperature data. That is, the body temperature calculating unit 222 may correct the body temperature data on the basis of pre-calibration data for the temperature sensor 130 of the patch-type thermometer 100. Here, the calibration data may be data related to the driving chip 121 of the patch-type thermometer 100.

More specifically, the body temperature calculating unit 222 may sequentially perform correction for internal resistance of the driving chip 121 of the patch-type thermometer 100, correction for an error (an RF harvesting error) that occurs when a voltage is rectified according to being inductively coupled by NFC tagging, and correction for the thermistor of the patch-type thermometer 100 and external resistance of the driving chip 121 to correct the body temperature data.

Here, a change in internal resistance of the driving chip affects a measurement value (Ch2ADC) for the thermistor and a measurement value (Ch3ADC) for external resistance. Therefore, the correction for the internal resistance of the driving chip is reflected in the measurement value for the thermistor and the measurement value for the external resistance.

In this case, the body temperature calculating unit 222 may perform correction by dividing the change characteristic of the measurement value of the ADC for the internal resistance into three sections. Here, each section may be a section that may be linearized. That is, the body temperature calculating unit 222 may determine a section for measurement values of the ADC corresponding to correction values (Ch2offset, Ch3offset) of the internal resistance of the driving chip and correct the measurement value (Ch2ADC) for the thermistor and the measurement value (Ch3ADC) for the external resistance by being linearized with a different slope for each section.

The RF harvesting affects the measurement value of the rectified voltage by NFC tagging and the external resistance. In this case, the body temperature calculating unit 222 may determine whether the measurement value of the rectified voltage by NFC tagging is out of a predetermined range or whether an absolute value of a difference between the measurement value (Ch3ADC) for the external resistance and the correction value (Ch3offset) of the external resistance is greater than or equal to a predetermined value, and when it is determined that the absolute value is greater than or equal to the predetermined value, the terminal 200 may determine that the value is invalid and re-measure the body temperature, and thus may perform correction for the RF harvesting error. Here, the determination of the RF harvesting error requires the measurement value for the external resistance and thus may be performed after the correction for the internal resistance of the driving chip.

Changes in thermistor and external resistance affect the measurement value (Ch2ADC) for the thermistor and the calculated body temperature value. Therefore, the correction for the thermistor and the external resistance is reflected in the measurement value (Ch2ADC) for the thermistor and a formula offset (β). In this case, a temperature value (Cla_C) at the time of calibration is also corrected. Here, the temperature value at the time of calibration is corrected by a predetermined correction value and a proportional constant.

First, the body temperature calculating unit 222 may add the correction value of the measurement value (Ch2ADC) for the thermistor due to the internal resistance to the correction value (Ch3offset) for the internal resistance of the driving chip for the measurement of the external resistance and subtract the correction value of the measurement value (Ch3ADC) for the external resistance due to the internal resistance from a result of the addition and thus may correct the measurement value (Ch2ADC) for the thermistor.

Further, the body temperature calculating unit 222 may multiply a difference between the correction value of the measurement value (Ch2ADC) for the thermistor according to the change in the thermistor and the correction value (Ch2offset) for the internal resistance of the driving chip for the thermistor measurement by the linearization constant (a) to add a correction constant (K) to a result of the multiplication, and subtract a result vale of the addition from the correction value of the temperature value (Cal_C) at the time of calibration and thus may correct the formula offset (β).

Further, as described above, the body temperature calculating unit 222 may calculate the body temperature value on the basis of the corrected body temperature data. Here, the body temperature value may be physical temperature information that the user may recognize.

In this case, the body temperature calculating unit 222 may calculate the body temperature value by multiplying the corrected body temperature data by the linearization constant (α) and summing the correction constant (K) and the formula offset (β). For example, the terminal 200 may calculate the body temperature value according to Equation 2 below.

Body temperature value=α×(CH2ADC)+β+K [Equation 2]

Here, CH2ADC denotes a finally corrected measurement value for the thermistor.

Further, the body temperature calculating unit 222 may determine whether a deviation of the calculated five body temperature values exceeds a predetermined value, and when it is determined that the deviation does not exceed the predetermined value, for example, when it is determined that the deviation does not exceed 0.1° C., the body temperature calculating unit 222 may calculate an average value of the five body temperature values as a body temperature value.

Further, when it is determined that the deviation of the calculated body temperature values exceeds the predetermined value, for example, when it is determined that the deviation exceeds 0.1° C., the body temperature calculating unit 222 may delete a first one piece of data among the pieces of received body temperature data, re-request measurement of the body temperature from the patch-type thermometer 100 to update the body temperature data to new data, and recalculate the body temperature value.

In this case, the body temperature calculating unit 222 may control the calculated body temperature value to be displayed through the display unit 230.

The body temperature information processing unit 224 may control the body temperature values calculated by the body temperature calculating unit 222 to be transmitted to the body temperature management server 300. In this case, the body temperature information processing unit 224 may automatically transmit the body temperature value to the body temperature management server 300 whenever the body temperature is measured by the patch-type thermometer 100 or whenever the body temperature value is calculated by the body temperature calculating unit 222. Further, the body temperature information processing unit 224 may collectively transmit the stored body temperature values in a predetermined time unit to the body temperature management server 300.

Alternatively, the body temperature information processing unit 224 may control the calculated body temperature values to be stored in the storage unit 240. That is, the body temperature values calculated by the body temperature calculating unit 222 may be transmitted to the body temperature management server 300 and, at the same time, stored in the terminal 200.

Further, the body temperature information processing unit 224 may request a trend of a change in body temperature for the accumulatively stored body temperature values from the body temperature management server 300. That is, the body temperature information processing unit 224 may request a trend of a change in body temperature for each preset period from the body temperature management server 300. In this case, the body temperature information processing unit 224 may control the trend of the change in body temperature provided from the body temperature management server 300 to be displayed on the display unit 230.

Accordingly, it is possible to provide the trend of the change in body temperature for a preset period to a medical staff to present an accurate health condition of the user. Therefore, it is possible to provide specific information for determining the health condition of the user to the medical staff and allow the medical staff to accurately and rapidly determine the health condition. In addition, the medical staff may rapidly take action, which may shorten the treatment process.

Further, the body temperature information processing unit 224 may request a health condition for the measured body temperature value from the body temperature management server 300. That is, when the measured body temperature value is abnormal, the body temperature information processing unit 224 may request a health condition corresponding to the body temperature value from the body temperature management server 300 so that the guardian may check the health condition of the user.

Optionally, the body temperature information processing unit 224 may request an action corresponding to the health condition together with or in addition to the request for the health condition. For example, whenever the health condition is requested or when the health condition is bad because the body temperature value is abnormal, the body temperature information processing unit 224 may request an action corresponding to the above case from the body temperature management server 300.

In this case, the body temperature information processing unit 224 may control the health condition and the action provided from the body temperature management server 300 to be displayed on the display unit 230.

Accordingly, even when there is no specialized knowledge or when it is difficult to transfer the user to a medical facility, a temporary action may be rapidly performed. Therefore, it is possible to effectively and stably manage the health of the user.

The display unit 230 is a display provided in the terminal 200 and may display the measured body temperature value. Further, the display unit 230 may display at least one of trend of changes in body temperature, the health condition, and the action, which are provided from the body temperature management server 300 in response to the request of the body temperature information processing unit 224.

The storage unit 240 is a temporary storage unit or a permanent storage unit, which is provided in the terminal 200, and may store the body temperature values calculated by the body temperature calculating unit 222.

The second communication unit 250 may communicate with the body temperature management server 300 through long-distance communication. That is, the second communication unit 250 may communicate with the body temperature management server 300 via a wired and/or wireless communication network.

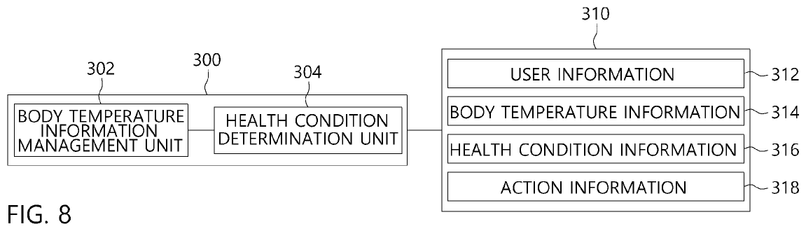

Referring to FIG. 8, the body temperature management server 300 may include a body temperature information management unit 302, a health condition determination unit 304, and a database 310.

The body temperature information management unit 302 stores and manages the body temperature values received from the terminal 200. In this case, the body temperature information management unit 302 controls the received body temperature values to be accumulated according to a measurement period of time and stored in the database 310.

Further, when the body temperature information management unit 302 receives the request for the trend of the change in body temperature from the terminal 200, the body temperature information management unit 302 may calculate a body temperature graph representing a trend of a change in body temperature for the corresponding user.

In this case, the body temperature information management unit 302 may calculate a body temperature graph for each preset time unit. For example, when a daily trend of the change in body temperature is requested, the body temperature information management unit 302 may calculate a graph representing the body temperature by hour or by minute. As another example, when a weekly trend of the change in body temperature is requested, the body temperature information management unit 302 may calculate a graph representing the body temperature by day or by time.

Further, the body temperature information management unit 302 may control the calculated body temperature graph to be transmitted to the terminal 200 through a communication unit (not illustrated).

The health condition determination unit 304 may determine a current health condition according to the body temperature value received from the terminal 200. Here, the determination of the health condition may be performed immediately upon receiving the body temperature value from the terminal 200 or may be performed in response to a separate request from the terminal 200.

In this case, the health condition determination unit 304 may determine the health condition, which corresponds to the measured body temperature value or to the body temperature value at the time of request, by searching health condition information 316 of the database 310.

Further, the health condition determination unit 304 may calculate an action corresponding to the determined health condition. Here, the calculation of the action may be performed upon receiving the request of the terminal 200, immediately upon determining the health condition, or upon determining that the health condition is abnormal after determining the health condition.

That is, the health condition determination unit 304 may calculate the action in response to the request of the terminal 200 or by itself. In this case, the health condition determination unit 304 may calculate the action corresponding to the health condition by searching action information 318 of the database 310.

Further, the body temperature information management unit 302 may control the determined health condition or the action to be transmitted to the terminal 200 through the communication unit (not illustrated).

The database 310 may include user information 312, body temperature information 314, the health condition information 316, and the action information 318.

The user information 312 may include user information and information about the patch-type thermometer 100. Here, the information about the patch-type thermometer 100 may be an UID of an NDEF. Further, the user information may include ID information about the terminal 200 of the user or guardian.

The body temperature information 314 may be body temperature information measured by the patch-type thermometer 100 and transmitted. Here, the body temperature information may include the body temperature value transmitted from the terminal 200 and the body temperature graph corresponding to the request.

The health condition information 316 may include health conditions for each body temperature. That is, the health condition information may include symptoms for each body temperature or related disease information.

The action information 318 may include actions for each health condition. Particularly, the action information may include information such as a temporary action corresponding to a state in which a health condition is degraded or an emergency action is required.

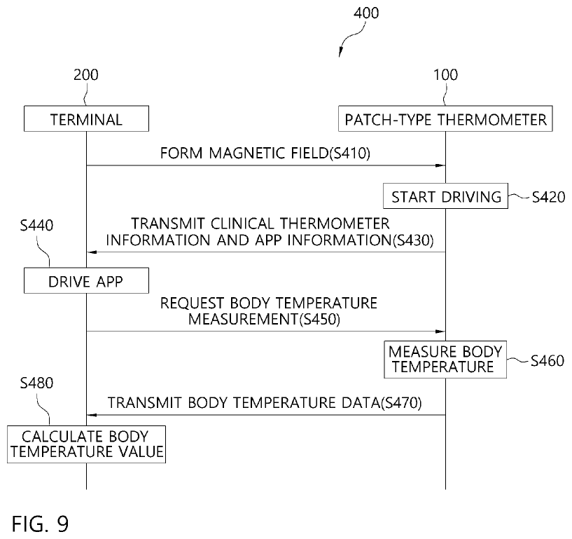

Hereinafter, patch-type thermometer-based body temperature management methods according to embodiments of the present invention will be described with reference to FIGS. 9 to 12.

A patch-type thermometer-based body temperature management method 400 according to an embodiment of the present invention relates to a method of measuring and calculating a temperature using a patch-type thermometer 100 when a temperature sensor 130 is an active sensor, and the patch-type thermometer-based body temperature management method 400 includes forming a magnetic field (S410), starting driving of the patch-type thermometer 100 (S420), transmitting clinical thermometer information and app information (S430), driving a related app (S440), requesting measurement of a body temperature (S450), measuring the body temperature (S460), transmitting body temperature data (S470), and calculating a body temperature value (S480), as illustrated in FIG. 9.

More specifically, first, a terminal 200 forms a magnetic field by being tagged to the patch-type thermometer 100 (in operation S410). In this case, the terminal 200 may be tagged to the patch-type thermometer 100 which is being attached to a body of a user. Further, the terminal 200 may form the magnetic field using the patch-type thermometer 100 by turning on an NFC function.

Next, the patch-type thermometer 100 starts to be driven by being inductively coupled with the magnetic field formed in the terminal 200 (in operation S420). In this case, the patch-type thermometer 100 may generate power for driving a driving chip 121 by being inductively coupled thereto.

Next, the patch-type thermometer 100 transmits the clinical thermometer information and the related app information to the terminal 200 (in operation S430). Here, the clinical thermometer information may be an UID of an NDEF. Further, the app information may include information about a body temperature management app or app store information about the corresponding app.

Next, the terminal 200 drives the corresponding app according to the related app information received from the patch-type thermometer 100 (in operation S440). In this case, the terminal 200 may start the corresponding app that is installed in advance. Alternatively, when the corresponding app is not installed in the terminal 200, the terminal 200 may be automatically linked to the corresponding app store.

Next, the terminal 200 requests measurement of the body temperature from the patch-type thermometer 100 (in operation S450). Here, although the terminal 200 is illustrated and described as requesting measurement of the body temperature after the corresponding app is driven, the present invention is not limited thereto, and the request for measurement of the body temperature may be performed simultaneously with the first magnetic field formation process.

Next, the patch-type thermometer 100 measures the body temperature of the user in response to the request of the terminal 200 (in operation S460). In this case, the patch-type thermometer 100 may use the driving chip 121 to drive the temperature sensor 130 by being inductively coupled with the magnetic field formed in the terminal 200 and measure the body temperature of the user.

Next, the patch-type thermometer 100 transmits the measured body temperature data to the terminal 200 (in operation S470). In this case, the patch-type thermometer 100 may transmit the body temperature data through a data field of the NDEF.

Next, the terminal 200 calculates the body temperature data transmitted from the patch-type thermometer 100 as a body temperature value (in operation S480). Here, the body temperature data may be data transmitted according to a data format, and the body temperature value may be physical temperature information that may be recognized by the user. That is, the terminal 200 may convert the body temperature data of a 16-bit value (unsigned integer) into physical scale data to calculate the body temperature value. For example, the terminal 200 may calculate the body temperature value according to Equation 1 above.

In this case, the calculated body temperature value may be displayed on the terminal 200. Optionally, the terminal 200 may store the calculated body temperature value in the storage unit 240.

Accordingly, the body temperature may be measured simply and rapidly without separate manipulation only by the terminal being tagged to the patch-type thermometer.

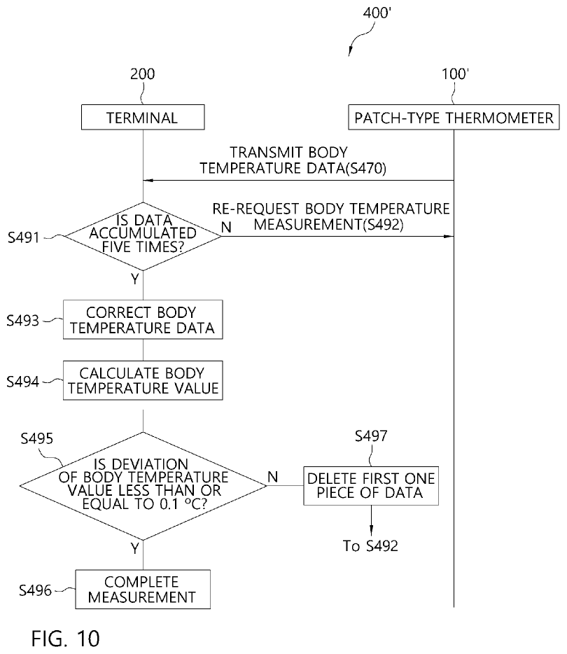

Meanwhile, a patch-type thermometer-based body temperature management method 400′ according to an embodiment of the present invention relates to a method of measuring and calculating a temperature using a patch-type thermometer 100′ when a temperature sensor 130 is a passive sensor, and the patch-type thermometer-based body temperature management method 400′ includes accumulating data five times (S491 and S492), correcting the data (S493), calculating a body temperature value (S494), and re-measuring the body temperature (S495 and S497) according to a result of the calculating, as illustrated in FIG. 10.

More specifically, first, when a terminal 200 is tagged to the patch-type thermometer 100′, the patch-type thermometer 100′ measures body temperature data and transmits the measured body temperature data to the terminal 200 (in operation S470). Since the above operation is the same as the operation described with reference to FIG. 9, a detailed description thereof will be omitted.

Next, the terminal 200 determines whether the body temperature data is accumulated a predetermined number of times (in operation S491). For example, the terminal 200 determines whether the body temperature data transmitted from the patch-type thermometer 100′ is accumulated five times, and when it is determined that the body temperature data is not accumulated five times, the terminal 200 re-requests measurement of the body temperature from the patch-type thermometer 100′ (in operation S492).

As a result of the determination in operation S491, when it is determined that the body temperature data is accumulated five times, that is, when the number of pieces of the body temperature data transmitted from the patch-type thermometer 100′ is five, the terminal 200 corrects the five pieces of body temperature data on the basis of calibration data predetermined for a temperature sensor of the patch-type thermometer 100′ (in operation S493). Here, the calibration data may be data related to a driving chip 121 of the patch-type thermometer 100′, as described above.

In this case, in the correction, correction for internal resistance of the driving chip of the patch-type thermometer 100′, correction for an error (an RF harvesting error) that occurs when a voltage is rectified according to being inductively coupled by NFC tagging, and correction for a thermistor and external resistance are performed sequentially.

First, a change in internal resistance of the driving chip affects a measurement value (Ch2ADC) for the thermistor and a measurement value (Ch3ADC) for the external resistance. Therefore, the correction for the internal resistance of the driving chip is reflected in the measurement value for the thermistor and the measurement value for the external resistance.

In this case, the terminal 200 performs correction by dividing the change characteristic of the measurement value of the ADC for the internal resistance into three sections. Here, each section may be a section that may be linearized. That is, the terminal 200 may determine a section for measurement values of the ADC corresponding to correction values (Ch2offset, Ch3offset) of the internal resistance of the driving chip and correct the measurement value (Ch2ADC) for the thermistor and the measurement value (Ch3ADC) for the external resistance by being linearized with a different slope for each section.

The RF harvesting affects the measurement value of the rectified voltage by NFC tagging and affects the external resistance. In this case, the terminal 200 may determine whether the measurement value of the rectified voltage by NFC tagging is out of a predetermined range or whether an absolute value of a difference between the measurement value (Ch3ADC) for the external resistance and the correction value (Ch3offset) of the external resistance is greater than or equal to a predetermined value, and when it is determined that the absolute value is greater than or equal to the predetermined value, the terminal 200 may determine that the value is invalid and re-measure the body temperature and thus may perform correction for the RF harvesting error. Here, the determination of the RF harvesting error requires the measurement value for the external resistance and thus is performed after the correction for the internal resistance of the driving chip.

Changes in thermistor and external resistance affect the measurement value (Ch2ADC) for the thermistor and the calculated body temperature value. Therefore, the correction for the thermistor and the external resistance is reflected in the measurement value (Ch2ADC) for the thermistor and a formula offset (β). In this case, a temperature value (Cla_C) at the time of calibration is also corrected. Here, the temperature value at the time of calibration is corrected by a predetermined correction value and a proportional constant.

First, the terminal 200 may add the correction value of the measurement value (Ch2ADC) for the thermistor due to the internal resistance to the correction value (Ch3offset) for the internal resistance of the driving chip for the measurement of the external resistance and subtract the correction value of the measurement value (Ch3ADC) for the external resistance due to the internal resistance from a result of the addition and thus may correct the measurement value (Ch2ADC) for the thermistor.

The terminal 200 may multiply a difference between the correction value of the measurement value (Ch2ADC) for the thermistor according to the change in the thermistor and the correction value (Ch2offset) for the internal resistance of the driving chip for the thermistor measurement by the linearization constant (α) to add a correction constant (K) to a result of the multiplication, and subtract a result vale of the addition from the correction value of the temperature value (Cal_C) at the time of calibration, and thus may correct the formula offset (β).

Next, the terminal 200 calculates the body temperature value on the basis of the corrected body temperature data (in operation S494). Here, the body temperature value may be physical temperature information that the user may recognize.

In this case, the terminal 200 may calculate the body temperature value by multiplying the corrected body temperature data by the linearization constant (α) and summing the correction constant (K) and the formula offset (β). For example, the terminal 200 may calculate the body temperature value according to Equation 2 above.

Next, the terminal 200 determines whether a deviation of the calculated body temperature values is less than a predetermined value (in operation S495). For example, the terminal 200 determines whether the deviation of the calculated body temperature value is less than or equal to 0.1° C., and when it is determined that the deviation of the body temperature value is 0.1° C., the measurement is completed (in operation S496).

In this case, the terminal 200 may calculate an average value of the five body temperature values as a body temperature value. Here, the calculated body temperature values may be displayed on the terminal 200. Optionally, the terminal 200 may store the calculated body temperature values.

As a result of the determination in operation S495, when it is determined that the deviation of the body temperature value exceeds 0.1° C., a first one piece of data among the pieces of the body temperature data transmitted from the patch-type thermometer 100′ is deleted (in operation S497). In this case, the process proceeds to operation S492 and the terminal 200 re-requests measurement of the body temperature from the patch-type thermometer 100′ to repeatedly perform operations S491 to S494 and updates the body temperature data to new data.

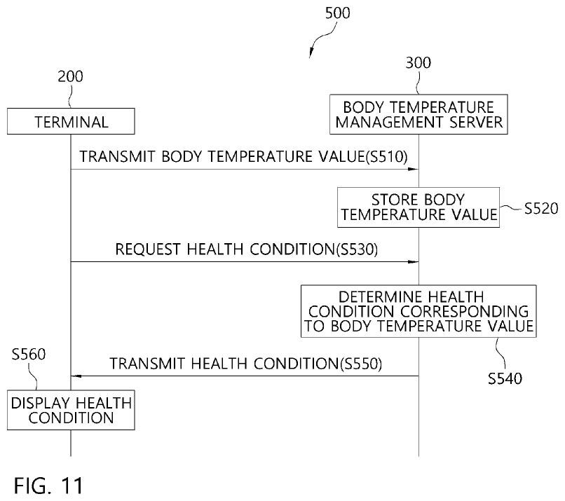

Meanwhile, a patch-type thermometer-based body temperature management method 500 according to an embodiment of the present invention includes transmitting a body temperature value (510), storing the body temperature value (S520), requesting a health condition (S530), determining the health condition according to the body temperature value (S540), transmitting the health condition (S550), and displaying the health condition (S560) as illustrated in FIG. 11.

More specifically, first, a terminal 200 transmits a calculated body temperature value to a body temperature management server 300 (in operation S510). In this case, the terminal 200 may automatically transmit the body temperature value to the body temperature management server 300 whenever the body temperature is measured by the patch-type thermometer 100 or the body temperature value is calculated.

Alternatively, the terminal 200 may collectively transmit the stored body temperature values in a predetermined time unit to the body temperature management server 300.

Accordingly, the measurement of the temperature using the patch-type thermometer may be performed and, at the same time, the transmission of the body temperature information may be performed. Therefore, there is no need for separate manipulation for management of the body temperature and thus convenience of use may be improved.

Next, the body temperature management server 300 stores and manages the body temperature values received from the terminal 200 (in operation S520). In this case, the body temperature management server 300 may accumulate the received body temperature values according to a measurement period of time to store the received body temperature values in a database 310.

Next, the terminal 200 requests a health condition corresponding to the body temperature value from the body temperature management server 300 (in operation S530). In this case, when the measured body temperature value is abnormal, the terminal 200 may request the health condition corresponding to the body temperature value from the body temperature management server 300 so that a guardian may check the health condition of the user.

Optionally, the terminal 200 may request an action corresponding to the health condition together with or in addition to the request for the health condition. For example, whenever the health condition is requested or when the health condition is bad because the body temperature value is abnormal, the terminal 200 may request an action corresponding to the above case from the body temperature management server 300.

Next, the body temperature management server 300 determines the health condition corresponding to the body temperature value (in operation S540). Here, the determination of the health condition may be performed immediately upon receiving the body temperature value from the terminal 200 or may be performed in response to a separate request from the terminal 200 as described above.

For example, the body temperature management server 300 may determine a current health condition corresponding to the body temperature value received from the terminal 200. As another example, the body temperature management server 300 may determine the health condition of the user according to a separate request from the terminal 200.

In this case, the body temperature management server 300 may determine the health condition, which corresponds to the measured body temperature value or to the body temperature value at the time of request, by searching health condition information 316 of the database 310 on the basis of the body temperature value.

Meanwhile, when a request for an action is received from the terminal 200, or immediately upon determination of the health condition, or when the body temperature management server 300 determines that the health condition is abnormal, an action according to the determined health condition may be calculated. That is, the body temperature management server 300 may calculate the action in response to the request of the terminal 200 or by itself. In this case, the body temperature management server 300 may calculate the measure corresponding to the health condition by searching action information 318 of the database 310.

Next, the body temperature management server 300 transmits the determined health condition to the terminal 200 (in operation S550). In this case, when the action corresponding to the health condition is calculated, the body temperature management server 300 may transmit the action together with the health condition to the terminal 200.

Next, the terminal 200 displays the health condition (in operation S560). In this case, when the body temperature management server 300 transmits the action together with the health condition, the terminal 200 may display the action together with the health condition.

Accordingly, even when there is no specialized knowledge or when it is difficult to transfer the user to a medical facility, a temporary action may be rapidly performed. Therefore, it is possible to effectively and stably manage the health of the user.

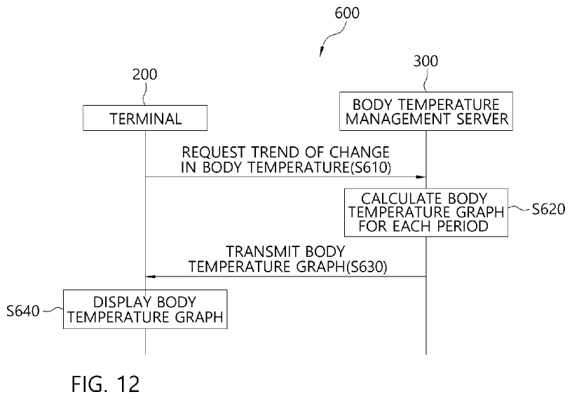

Meanwhile, a patch-type thermometer-based body temperature management method 600 according to an embodiment of the present invention includes requesting a trend of a change in body temperature (S610), calculating a body temperature graph (S620), transmitting the body temperature graph (S630), and displaying the body temperature graph (S640), as illustrated in FIG. 12.

More specifically, first, a terminal 200 requests a trend of a change in body temperature from a body temperature management server 300 (in operation S610). In this case, the terminal 200 may request a trend of a change in body temperature for a body temperature value accumulated in a database 310 for each preset period.

Next, the body temperature management server 300 calculates a body temperature graph for each period using the stored body temperature value (in operation S620). In this case, the body temperature management server 300 may calculate a body temperature graph for each preset time unit. For example, when a daily trend of a change in body temperature is requested, the body temperature management server 300 may calculate a graph representing the body temperature by hour or by minute. As another example, when a weekly trend of a change in body temperature is requested, the body temperature management server 300 may calculate a graph representing the body temperature by day or by time.

Next, the body temperature management server 300 transmits the calculated body temperature graph to the terminal 200 (in operation S630).

Next, the terminal 200 displays the body temperature graph transmitted from the body temperature management server 300 (in operation S640).

Accordingly, it is possible to provide the trend of the change in body temperature for a preset period to a medical staff to present an accurate health condition of the user. Therefore, it is possible to provide specific information for determining the health condition of the user to the medical staff and allow the medical staff to accurately and rapidly determine the health condition. In addition, the medical staff may rapidly take action, which may shorten the treatment process.

The above methods may be implemented by the terminal 200 and the body temperature management server 300 as illustrated in FIG. 1, and particularly, may be implemented as software programs that perform the above operations. In this case, the programs may be stored in a computer-readable recording medium or may be transmitted via a computer data signal that is combined with a carrier wave in a transmission medium or in a communication network.

In this case, the computer-readable recording medium may include any type of recording device in which data readable by a computer system is stored and may include, for example, a read only memory (ROM), a random access memory (RAM), a compact disc ROM (CD-ROM), a digital video disc ROM (DVD-ROM), a digital video disc RAM (DVD-RAM), a magnetic tape, a floppy disk, a hard disk, an optical data storage device, or the like.

While the embodiments of the present invention have been described, the spirit of the present invention is not limited to the embodiments presented in this specification. Those skilled in the art who understand the spirit of the present invention may easily suggest other embodiments by adding, changing, or deleting elements within the scope of the same concept, and the other embodiments are also within the spirit of the present invention.

1. A patch-type thermometer-based body temperature management system comprising:
a patch-type thermometer which is attached to skin of a user to measure body temperature and transmits a user identification (ID), related app information, and body temperature data on the basis of near-field communication (NFC);
a terminal configured to form a magnetic field by being tagged to the patch-type thermometer, request measurement of the body temperature, receive the user ID, the related app information, and the body temperature data from the patch-type thermometer, and calculate body temperature values; and
a body temperature management server configured to receive the body temperature values from the terminal, accumulate and store the received body temperature values, calculate at least one of a trend of a change in body temperature over time and a health condition corresponding to the received body temperature values, and transmit the calculated trend of the change or health condition to the terminal.

2. The patch-type thermometer-based body temperature management system of claim 1, wherein the terminal includes:
a body temperature calculating unit configured to calculate the body temperature values using the body temperature data received from the patch-type thermometer; and
a body temperature information processing unit configured to control the body temperature values to be transmitted to the body temperature management server, a trend of a change in body temperature for the accumulated body temperature values and the health condition to be requested to the body temperature management server, and corresponding information to be received and displayed.

3. The patch-type thermometer-based body temperature management system of claim 2, wherein the body temperature calculating unit receives a plurality of pieces of body temperature data from the patch-type thermometer,
the body temperature calculating unit performs correction for the plurality of pieces of body temperature data and then calculates the body temperature values according to the corrected body temperature data,
calculates an average value of the calculated body temperature values when a deviation of the calculated body temperature values is less than or equal to a predetermined value,
deletes a first one piece of data among the pieces of body temperature data,
updates the body temperature data to new data, and
re-calculates the body temperature value when the deviation of the calculated body temperature values exceeds the predetermined value.

4. The patch-type thermometer-based body temperature management system of claim 3, wherein the body temperature calculating unit corrects the body temperature data on the basis of pre-calibration data for a temperature sensor of the patch-type thermometer, and
the body temperature calculating unit sequentially performs correction for internal resistance of a driving chip of the patch-type thermometer, correction for an error (a radio frequency (RF) harvesting error) that occurs when a voltage is rectified according to being inductively coupled by NFC tagging, and correction for a thermistor of the patch-type thermometer and external resistance of the driving chip to correct the body temperature data.

5. The patch-type thermometer-based body temperature management system of claim 2, wherein the terminal includes:
a first communication unit configured to perform NFC with the patch-type thermometer;
a second communication unit configured to perform communication with the body temperature management server via a wired or wireless communication network;
a display unit configured to display the body temperature values and corresponding information received from the body temperature management server; and
a storage unit configured to store the body temperature values.

6. The patch-type thermometer-based body temperature management system of claim 1, wherein the body temperature management server includes:
a body temperature information management unit configured to accumulate, store, and manage the body temperature values received from the terminal over time, and calculate a body temperature graph according to the trend of the change in body temperature;
a health condition determination unit configured to determine a current health condition according to the received body temperature values; and
a database configured to store user information, body temperature information, and action information.

7. The patch-type thermometer-based body temperature management system of claim 6, wherein the body temperature information management unit calculates the body temperature graph for each preset time unit.

8. The patch-type thermometer-based body temperature management system of claim 6, wherein the health condition determination unit calculates an action corresponding to the determined health condition.

9. The patch-type thermometer-based body temperature management system of claim 1, wherein the patch-type thermometer includes:
a flexible circuit board having at least one surface on which an antenna pattern is formed and at least one driving chip is mounted;
a temperature sensor mounted on an upper surface of the flexible circuit board so as to measure the body temperature;
a heat transfer member which is electrically connected to the temperature sensor through a via hole and is mounted on a lower surface of the flexible circuit board so as to be in direct contact with the skin of the user; and
a protective member configured to surround the flexible circuit board so as to prevent the antenna pattern, the driving chip, and the temperature sensor from being exposed to the outside.

10. The patch-type thermometer-based body temperature management system of claim 1, wherein the patch-type thermometer is driven by being inductively coupled with the magnetic field.

11. A patch-type thermometer-based body temperature management method comprising:
forming a magnetic field by a terminal being tagged to a patch-type thermometer attached to a body of a user;
starting driving of the patch-type thermometer by being inductively coupled with the magnetic field;
transmitting, by the patch-type thermometer, a user identification (ID) and related app information to the terminal;
driving, by the terminal, a corresponding app according to the related app information;
measuring, by the patch-type thermometer, a body temperature of the user in response to a request of the terminal and transmitting body temperature data to the terminal; and
calculating, by the terminal, body temperature values according to the body temperature data.

12. The patch-type thermometer-based body temperature management method of claim 11, wherein:
the transmitting of the body temperature data to the terminal includes measuring the body temperature data multiple times and repeatedly transmitting the measure data; and
the calculating of the body temperature values includes performing correction on the body temperature data transmitted multiple times, then calculating the body temperature values according to the corrected body temperature data, calculating an average value of the calculated body temperature values when a deviation of the calculated body temperature values is less than or equal to a predetermined value, deleting a first one piece of data among the pieces of body temperature data, updating the body temperature data to new data, and re-calculating the body temperature value when the deviation of the calculated body temperature value exceeds the predetermined value.

13. The patch-type thermometer-based body temperature management method of claim 12, wherein:
the calculating of the body temperature values includes correcting the body temperature data on the basis of pre-calibration data for a temperature sensor of the patch-type thermometer; and
the correction of the body temperature data includes sequentially performing correction for internal resistance of a driving chip of the patch-type thermometer, correction for an error (a radio frequency (RF) harvesting error) that occurs when a voltage is rectified according to being inductively coupled by near-field communication (NFC) tagging, and correction for a thermistor of the patch-type thermometer and external resistance of the driving chip.

14. The patch-type thermometer-based body temperature management method of claim 11, further comprising:
transmitting, by the terminal, the calculated body temperature values to a body temperature management server; and
accumulating and storing, by the body temperature management server, the received body temperature values over time.

15. The patch-type thermometer-based body temperature management method of claim 14, further comprising:
requesting, by the terminal, a health condition corresponding to the body temperature value from the body temperature management server;
determining, by the body temperature management server, the health condition corresponding to the body temperature value; and
receiving, by the terminal, the health condition from the body temperature management server and displaying the health condition.

16. The patch-type thermometer-based body temperature management method of claim 15, wherein the determining of the health condition further includes calculating an action corresponding to the determined health condition, and
the displaying of the health condition includes displaying the action together with the health condition.

17. The patch-type thermometer-based body temperature management method of claim 14, further comprising:
requesting, by the terminal, a trend of a change in body temperature from the body temperature management server;
calculating, by the body temperature management server, a body temperature graph for each period using the stored body temperature values; and
receiving, by the terminal, the calculated body temperature graph from the body temperature management server and displaying the body temperature graph.

18. The patch-type thermometer-based body temperature management method of claim 17, wherein, the calculating of the body temperature graph includes calculating the body temperature graph for each preset time unit.
